(19) United States
(12) Patent Application Publication
(10) Pub. No.: US 2026/0116837 A1
(43) Pub. Date: Apr. 30, 2026

(54) METHOD FOR NON-CATALYTIC EXTRACTION OF PHENOLIC ALDEHYDES FROM LIGNOCELLULOSIC BIOMASS

(71) Applicant: PLINIUS LABS NV, Aalter (BE)

(72) Inventors: Bart ROSSEL, Aalter (BE); Kevin HUVAERE, Aalter (BE); Yves BOONEN, Aalter (BE)

(21) Appl. No.: 19/121,008

(22) PCT Filed: Oct. 17, 2023
(86) PCT No.: PCT/EP2023/078851

§ 371 Date: Apr. 14, 2025

(30) Foreign Application Priority Data
Oct. 17, 2022 (BE) 2022/5832
Oct. 17, 2022 (BE) 2022/5833

(51) Int. Cl. C07C 45/79; C07C 45/85; C07C 51/47; C07C 51/487

(52) U.S. Cl. CPC C07C 45/79; C07C 45/85; C07C 51/47; C07C 51/487

(57) ABSTRACT

The current invention relates to a Method for extracting phenolic aldehydes from a lignocellulosic biomass comprising the steps of: (i) mechanochemically reacting said lignocellulosic biomass with a dry base, thereby agglomerating said lignocellulosic biomass with said dry base under pressure to form agglomerated bodies; and (ii) oxidative processing of the agglomerated bodies, comprising the step of boiling said agglomerated bodies for a period of time sufficient to extract phenolic aldehydes from said agglomerated bodies, wherein said lignocellulosic biomass is derivable from one or more herbaceous crops, and wherein said dry base is a dry inorganic oxide, a dry inorganic hydroxide, or a mixture thereof.

### FIELD OF THE INVENTION

The present invention relates to a method for extracting phenolic aldehydes from a lignocellulosic biomass. In another aspect, the present invention also relates to natural phenolic aldehydes.

### BACKGROUND

Recently, mechanochemistry has attracted much attention because it allows promotion of reactions under solvent-free conditions.

EP2964600 discloses a method for the production of an oxidized reaction product, comprising the step of mechanocatalytically reacting an amount of a polymer containing material and an oxidation catalyst, the oxidation catalyst being a solid metal oxide comprising at least one of manganese oxides, cerium oxides, copper oxides, or silver oxides.

A mechanochemical approach for the cleavage of β-O-4-linkages in lignin is reported by Tillmann Kleine et al. in DOI:10.1039/C2GC36456E. The method comprises solvent-free ball milling of pure lignin and beech wood in the presence of a base for lignin degradation for 12 hours.

These known methods have multiple disadvantages or problems such as long reaction times, the use of catalysts, and difficult dust control.

When phenolic aldehydes are extracted for food purposes, for example as flavours, traditional methods for extraction of phenolic aldehydes fall short in that they are not natural because they involve a feedstock contaminated by sulphite from paper pulping and chemical catalysts.

The aim of the invention is to provide an extraction method which eliminates those disadvantages. The invention thereto aims to provide a method for a non-catalytic extraction of phenolic aldehydes from lignocellulosic biomass.

### SUMMARY OF THE INVENTION

The present invention and embodiments thereof serve to provide a solution to one or more of above-mentioned disadvantages. To this end, the present invention relates to a method for extracting phenolic aldehydes from lignocellulosic biomass according to claim 1. Preferred embodiments of the method are shown in any of the claims 2 to 13. In a second aspect, the present invention also relates phenolic aldehydes according to claim 14.

The method according to the invention is advantageous because phenolic aldehydes can be extracted using physical extraction, without the use of catalysts or oxidative chemicals.

The inventors have unexpectedly observed that by agglomerating lignocellulosic biomass with a dry base, the lignocellulosic biomass undergoes macroscopic modification under the pressure and heat of the agglomerated bodies, which results in improved availability and accessibility of the ligneous material in the extraction phase.

Furthermore, the method has the following advantages: agglomerating spontaneously cuts vanillin from the lignin polymer present in the lignocellulosic biomass; it is a convenient way to process the dusty lignocellulosic biomass; it makes a perfect suspension with the base; and it provides an intimate contact surface between the biomass and hydroxide and/or oxide prior to the extraction phase.

By agglomerating the lignocellulosic biomass in a dry pretreatment step, the lignocellulosic biomass showed superior wettability and gave ideal release conditions for subsequent pressure cooking.

The dry agglomeration ensures that the process temperature in the extraction phase can be reduced and work at a relatively mild temperature (max 120° C.) with respect to other lignin-to-monomer extractive procedures.

The method according to the current invention is also advantageous because the mild oxidative processing to extract phenolic aldehydes will preserve most of the chemical structures (cellulose, lignin oligomers most likely not condensed) suited for further extraction or modification.

In a further preferred embodiment, the inventors have unexpectedly observed that agglomerating lignocellulosic biomass with a dry inorganic oxide, a dry inorganic hydroxide, or a mixture thereof, improves subsequent extraction of phenolic aldehydes from said lignocellulosic biomass. The dry pretreatment according to the invention improves availability and accessibility of ligneous material present in the lignocellulosic biomass. This pretreatment ensures that a subsequent extraction can be carried out at a relatively mild temperature with respect to other lignin-to-monomer extractive procedures.

### DETAILED DESCRIPTION OF THE INVENTION

The present invention concerns a method for extracting phenolic aldehydes from a lignocellulosic biomass.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“Base” as used herein refers to a substance capable of accepting or neutralizing hydrogen ions. “Alkali” as used herein refers to a base that dissolves in water.

“Dry” as used in herein refers to a substance which is in a solid state of matter or non-aqueous phase with a low moisture content such as a moisture content lower than 10% by weight, preferably lower than 9% by weight, preferably lower than 8% by weight, preferably lower than 7% by weight, preferably lower than 6% by weight, preferably lower than 5% by weight.

“Solid” as used herein refers to the solid state of matter, wherein the molecules in a solid are closely packed together and contain the least amount of kinetic energy.

“Agglomeration” as used in herein refers to the mechanical process of compressing primary solid particles or solid material under pressure to form larger multiparticle entities.

“Oxidative processing” as used herein refers to those chemical/biochemical reactions that involve oxygen or air as oxidant. During oxidative processing lignin building blocks, such as p-coumaryl alcohol, coniferyl alcohol, and sinapyl alcohol, are selectively cleaved from the lignocellulosic biomass by an oxidant resulting in phenolic aldehydes.

Phenolic aldehydes as used herein refers to both their free form as well as the salt form. In alkaline environments, phenolic aldehydes typically occur in their salt form. Preferably, the extracted phenolic aldehydes refer to phenolic aldehydes in their free form as well as salts thereof.

“Agglomerated body” as used herein refers to a multiparticle entity formed when primary solid particles or solid material are compressed under pressure and are made to adhere to form larger multiparticle entities, examples of agglomerated bodies are pellets, extrudates, granulates, granules, tablets, and the like.

“A”, “an”, and “the” as used herein refers to both singular and plural referents unless the context clearly dictates otherwise. By way of example, “a base” refers to one or more than one base.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any 3, 4, 5, 6 or 7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In a first aspect, the invention provides a method for extracting phenolic aldehydes from a lignocellulosic biomass.

In another embodiment of the first aspect, the invention provides a method for extracting at least one compound selected from the list of: vanillin, syringaldehyde, apocynin, vanillic acid, acetosyringone, syringic acid and phenolic aldehydes. More preferably, the invention provides a method for extracting at least one compound selected from the list of: vanillin, syringaldehyde, apocynin, vanillic acid, acetosyringone and syringic acid. Most preferably, the invention provides a method for extracting at least one compound selected from the list of: vanillin, apocynin, vanillic acid, or a mixture thereof.

In a particularly preferred embodiment, the method comprises the steps of:
i. mechanochemically reacting said lignocellulosic biomass with a dry base, thereby agglomerating said lignocellulosic biomass with said dry base under pressure to form agglomerated bodies; and
ii. oxidative processing of the agglomerated bodies, comprising the step of boiling said agglomerated bodies for a period of time sufficient to extract phenolic aldehydes from said agglomerated bodies.

In a further embodiment, the dry base is a dry inorganic oxide, a dry inorganic hydroxide, or a mixture thereof.

The inventors have unexpectedly observed that by agglomerating lignocellulosic biomass with a dry base, the lignocellulosic biomass undergoes macroscopic modification under the pressure and heat of the agglomerated bodies, which results in improved availability and accessibility of the ligneous material during oxidative processing.

The dry agglomeration ensures that the process temperature during oxidative processing can be reduced and be carried out at a relatively mild temperature with respect to other lignin-to-monomer extractive procedures.

The term “lignocellulosic biomass”, as used in the text, refers to plant biomass containing cellulose, hemicelluloses, and lignin. Typically, in such a lignocellulosic biomass, the cellulose, hemicellulose, and lignin are bound together in a complex macromolecular structure along with small quantities of extractives, pectin, protein, and ash. A substantial benefit of the presently disclosed and/or claimed inventive concept(s) is that the lignin does not have to be separated away from the cellulose and/or hemicellulose prior to the pretreatment, nor the possible subsequent extraction, thereby eliminating a significant portion of the waste component from the process and eliminating the need to purify the lignocellulosic biomass before pretreatment of extraction. Any quantity of lignin-containing material may be provided and used in the presently disclosed and/or claimed inventive concept(s).

The nature or origin of the lignocellulosic biomass should not be considered to be constraining to the processes and methods disclosed herein, i.e., the lignocellulosic biomass is source and composition independent and one of ordinary skill in the art, given the present disclosure, would appreciate that the origin and composition of the lignocellulosic biomass could be tailored or blended in such a manner to provide any number of different phenolic aldehydes using the conditions disclosed herein. Indeed, the inventors have found to date that a wide variety of lignin-containing materials that have been tested are suitable and appropriate for the processes and methods disclosed herein.

During mechanochemical reaction in step (i), the dry base functions in the same phase as the lignocellulosic biomass and, pursuant to the presently disclosed concept(s), the dry base is in the solid phase as is the lignocellulosic biomass.

In a preferred embodiment, the lignocellulosic biomass is derivable from one or more herbaceous crops. In an even more preferred embodiment, the lignocellulosic biomass is one or more herbaceous crops.

The terms “herbaceous crops” and “herbaceous plants”, as used in the text, are synonyms and refer to vascular plants that have no persistent woody stems above ground. These can be perennial herbaceous plants, annual herbaceous plants, or biennial herbaceous plants.

In a further preferred embodiment, the herbaceous crop is chosen from the list of annual herbaceous crops, biennial herbaceous crops, or a mixture thereof. In an even more preferred embodiment, the herbaceous crop is chosen from the list of: Linum sp. (flaxes), Poaceae sp. (grasses), Cannabis sp (Hemp), or a mixture thereof.

Examples of grasses are: barley; maize; oats; rice; rye; sorghum; wheat; millet; bamboo; marram grass; meadow-grass; reeds; ryegrass; sugarcane; bahiagrass; bentgrass; bermudagrass; bluegrass; buffalograss; centipede grass; fescue ryegrass; st. Augustine grass; zoysia; calamagrostis spp.; cortaderia spp.; deschampsia spp.; festuca spp.; melica spp.; muhlenbergia spp.; stipa spp. Grasses also includes processed grasses or byproducts thereof such as straw, which is defined as the dry stalks of cereal plants after the grain and chaff have been removed.

Examples of flaxes are: Linum africanum; Linum alatum (winged flax); Linum album; Linum alpinum; Linum arboreum (tree flax); Linum arenicola (sand flax); Linum aristatum (bristle flax); Linum australe (southern flax); Linum austriacum (Asian flax); Linum berlandieri (Berlandier's yellow flax); Linum bienne (pale flax); Linum campanulatum; Linum cariense; Linum carteri (Carter's flax); Linum catharticum (fairy flax); Linum compactum (Wyoming flax); Linum cratericola (Galepagos Islands flax); Linum dolomiticum; Linum elongatum (Laredo flax); Linum flavum (golden flax); Linum floridanum (Florida yellow flax); Linum grandiflorum (scarlet flax, flowering flax); Linum hirsutum (downy flax); Linum hudsonioides (Texas flax); Linum imbricatum (tufted flax); Linum intercursum (sandplain flax); Linum kingii (King's flax); Linum leoni; Linum lewisii (Lewis' blue flax, Lewis flax); Linum usitatissimum; Linum lundellii (Sullivan City flax); Linum macrocarpum (Spring Hill flax); Linum marginale (Australian native flax); Linum medium (stiff yellow flax); Linum monogynum (New Zealand linen flax); Linum narbonense (blue flax); Linum neomexicanum (New Mexico yellow flax); Linum perenne (perennial blue flax); Linum pratense (meadow flax); Linum puberulum (plains flax); Linum pubescens; Linum rigidum (stiffstem flax); Linum rupestre (rock flax); Linum schiedeanum (Schiede's flax); Linum strictum (ridged yellow flax); Linum subteres (Sprucemont flax, slenderfoot flax); Linum suffruticosum; Linum sulcatum (grooved flax); Linum tenuifolium; Linum trigynum (French flax); Linum ucranicum; Linum usitatissimum (common cultivated flax); Linum vernal (Chihuahuan flax); Linum virginianum (woodland flax); Linum westii (West's flax).

Examples of cannabis sp. are: Cannabis sativa; Cannabis indica; Cannabis ruderalis Janisch.

In a particularly preferred embodiment, the lignocellulosic biomass is flax, straw, or a mixture thereof. The heterogeneous nature of flax shives and straw (such as wheat straw) severely complicates use in available reactor systems. Different length of stalks, dust formation, the tendency to clog pipes or transfer lines, and the poor wettability were often heard concerns. The invention provides a valuable valorization for these difficult to handle crops.

Although the lignocellulosic biomass and/or the dry base may have an inherent moisture content, either inherently at harvest or due to wetting of the biomass to control dust or heating, to aid compaction, to improve internal transport, storage or for any other processing reason, it should be understood that the reactants, either alone or in combination, are still to be considered in a solid or non-aqueous phase. It should be understood, however, that the existence of such an amount of humidity in the components should not be interpreted to mean that the agglomeration occurs in an aqueous phase: rather, while some minor amount of water may be present inherently or from said wetting measures, the mechanochemical reaction between the lignocellulosic biomass and the dry base is carried out in a non-aqueous (and solvent-free) phase and the lignocellulosic biomass and the dry base should be understood to be in a solid form.

In an embodiment, the lignocellulosic biomass has a moisture content of maximum 25% by weight prior to step (i). Preferably, the moisture content is maximum 20% by weight prior step (i), more preferably maximum 15% by weight prior to step (i).

Too much moisture causes a paste to form and clogs the die. In an embodiment, it may be needed to dry certain types of lignocellulosic biomasses, for example in rotary drum dryers, to reduce the moisture content.

In another embodiment, it may be needed to add wetting agents to control dust or heating, to aid compaction, to improve internal transport, storage or for any other processing reason. Wetting agents, such as water, may be added in an amount of up to 20% by weight of the total of lignocellulosic biomass. Preferably wetting agents are added in an amount of up to 15% by weight, and more preferably in an amount of up to 10% by weight.

In such embodiments, the resulting wetted lignocellulosic biomass can have a moisture content of between 10 and 30% by weight prior to the step of agglomeration, preferably the resulting wetted lignocellulosic biomass can have a moisture content of between 15 and 30% by weight prior to the step of agglomeration, or even between 20 and 25% by weight prior to the step of agglomeration.

The terms “moisture content” and “water content”, as used in the text, are synonyms and refer to the quantity of water contained in the lignocellulosic biomass or in the dry base expressed in % by weight of the total component at ambient temperature and ambient pressure. The moisture content can be measured by techniques known in the art, such as oven-dry method and moisture meter method.

The ratio of the lignocellulosic biomass to the dry base is in step (i) such that the depolymerization of the lignin and the release of the phenolics aldehyde is optimized. Generally, the efficiency is optimized by determining a ratio, wherein a surface interaction of lignocellulosic biomass and the dry base is maximized and the release of specified or targeted phenolic aldehydes is optimized. In one embodiment, but not by way of limitation, said lignocellulosic biomass is agglomerated with said dry base in a ratio by weight between 5/0.1 and 5/5, preferably in a ratio by weight between 5/0.25 and 5/4.5, more preferably in a ratio by weight between 5/0.5 and 5/4, even more preferably in a ratio by weight between 5/0.75 and 5/3.5, even more preferably in a ratio by weight between 5/1 and 5/3.

The inventors unexpectedly observed that adding said dry inorganic oxide, said dry inorganic hydroxide, or said mixture thereof to the lignocellulosic biomass and simultaneous agglomeration results in softer agglomerated bodies, which dissolve easier during oxidative processing in step (ii). Furthermore, addition of said dry base raises the surface temperature of the agglomerated bodies, which is needed to release the phenolic aldehydes from said lignocellulosic biomass.

The inventors observed that if too little of said dry base is added to said lignocellulosic biomass, the surface temperature of the agglomerated bodies is not raised enough.

The inventors observed that if too much of said dry inorganic oxide, said dry inorganic hydroxide, or said mixture thereof is added to said lignocellulosic biomass, the obtained agglomerated bodies will be too soft and will crumble. Furthermore, too much of said dry inorganic oxide, said dry inorganic hydroxide, or said mixture thereof can lead to high surface temperatures which can cause charring.

In a preferred embodiment, the surface temperature of said agglomerated bodies is higher than 70° C., preferably higher than 75° C., more preferably higher than 80° C., even more preferably higher than 85° C., even more preferably higher than 90° C., even more preferably higher than 95° C., even more preferably higher than 100° C., even more preferably higher than 105° C., even more preferably higher than 110° C.

A minimum surface temperature is needed for the phenolic aldehydes to be released from the lignocellulosic biomass in the agglomerated bodies.

In another or further embodiment, the surface temperature of said agglomerated bodies is lower than 180° C., preferably lower than 175° C., more preferably lower than 170° C., even more preferably lower than 165° C., even more preferably lower than 160° C., even more preferably lower than 155° C., even more preferably lower than 150° C., even more preferably lower than 145° C., even more preferably lower than 140° C. It was found that surface temperatures that were too high resulted in charring of the agglomerated bodies and gave rise to significant delignification and decomposition.

In another or further embodiment, the surface temperature of said agglomerated bodies is between 7° and 180° C., preferably between 75 and 175° C., more preferably between 8° and 170° C., even more preferably between 85 and 165° C., even more preferably between 9° and 160° C., even more preferably between 95 and 155° C., even more preferably between 10° and 150° C., even more preferably between 105 and 145° C., even more preferably between 11° and 140° C.

The heat and pressure that the agglomeration generates, plasticizes the lignin content and leads to improved contact between the base which promotes the digestion, solubility, and oxidative processing of the agglomerated bodies in step (ii).

In an embodiment, the agglomerated body as a pH above or equal to 12. The alkaline environment is believed to improve the release of phenolic aldehydes from the lignocellulosic biomass.

In a preferred embodiment, the release of phenolic aldehydes includes both phenolic aldehydes in their free form as well as salts thereof. In alkaline conditions, such as a pH above 12, the phenolic aldehydes are predominantly released in salt form.

In an embodiment, said dry base is a dry alkali. In a preferred embodiment said dry base is chosen from the list of: dry sodium hydroxide (NaOH), dry potassium hydroxide (KOH), dry calcium hydroxide (Ca(OH)₂), dry calcium oxide (CaO), dry magnesium hydroxide (Mg(OH)₂), dry magnesium oxide (MgO), or a mixture thereof, preferably dry NaOH, dry KOH, or a mixture thereof.

Alternatively, an inorganic carbonate can be used for agglomeration with the lignocellulosic biomass, such as dry sodium carbonate (Na₂CO₃), or potassium carbonate (K₂CO₃).

The dry base is in a solid phase, such as a powder, pellets, flakes, granules, granulates, extrudates, and the like. Preferably the dry base is in the form of pellets or granulates.

In a preferred embodiment, said lignocellulosic biomass and said dry base are agglomerated in step (i) by compressing them through a die.

The inventors found that agglomerating the lignocellulosic biomass with dry inorganic oxide, dry inorganic hydroxide, or a mixture thereof, generates pressure by increasing friction in the die.

In a first particularly preferred embodiment, said lignocellulosic biomass and said dry base are pelletized during agglomerating in step (i). The term “pelletizing”, as used herein, is the process of compressing or molding a material into the shape of a pellet. In a further embodiment, the resulting agglomerated bodies are rounded, spherical, or cylindrical pellets.

In a further embodiment, the pelletizing is carried out by compressing the lignocellulosic biomass and the dry base through a die. The die can be a flat die or a ring die.

Flat dies are preferred because they are smaller in size and lightweight, easier to maintain and clean due to ready disassembly.

Ring dies are preferred because they have a high capacity, less wear and higher durability, and lower energy consumption.

In a further embodiment, the step of pelleting is repeated at least once, preferably at least twice. The inventors have found that sequential pelleting steps increase the surface temperature and contact pressure of the pellet which results in higher amounts of plasticized lignin, more energy in the biomass pellet, and leads to improved contact between the lignocellulosic biomass and the dry base which promotes further the digestion, oxidation, and solubility of lignocellulosic biomass.

In a further embodiment, the pellet dies have hole diameters of between 1 and 20 mm, preferably of between 2 and 15 mm, even more preferably of between 3 and 12 mm. In another or an even further embodiment, the pellet dies have a stroke length of between 5 and 100 mm, preferably of between 10 and 80 mm. In another or an even further embodiment, the ratio of the stroke length to the hole diameter is at least 0.5, preferably at least 0.8, more preferably at least 1. In another or an even further embodiment, the ratio of the stroke length to the hole diameter is at most 100, preferably at most 50, more preferably at most 30. In another or an even further embodiment, the ratio of the stroke length to the hole diameter lies between 0.5 and 100, preferably between 0.8 and 50, more preferably between 1 and 30. The inventors have found that the relation of hole diameter to stroke length of the die holes is inversely proportional to the energy content of the lignocellulosic biomass, and that a high ratio of stroke length to hole diameter significantly increases the digestion, oxidation, and solubility of lignocellulosic biomass.

In a second particularly preferred embodiment, said lignocellulosic biomass and said dry base are extruded during agglomeration in step (i).

In this embodiment, said lignocellulosic biomass and said dry base may be grinded and mixed prior to extrusion, combined with subsequent extrusion of the mixed product, or said lignocellulosic biomass and said dry base may be co-extruded.

The terms “extrusion” and “extrude”, as used in the text, refer to the process of pushing one or more materials through a single die of the desired cross-section to create objects of a fixed cross-sectional profile.

The terms “co-extrusion” and “co-extrude”, as used in the text, refer to the process of pushing two or more materials through a single die of the desired cross-section to create objects of a fixed cross-sectional profile.

Extrusion has the advantage that higher shear forces can be reached. Extrusion also allows for longer and smaller agglomerated bodies that can be obtained. Furthermore, extrusion allows for more pressure and temperature control.

In an embodiment, the lignocellulosic biomass can be processed or cleaned prior to agglomeration in step (i) to eliminate contaminants such as stones, for example with a destoner, and metal particles, for example by magnetic clearing. It can be important depending on the material's origin, as to prevent mechanical failure of the agglomeration equipment.

In some embodiments, the lignocellulosic biomass is milled prior to the agglomeration steps, for example to reduce the size of the lignocellulosic biomass.

In a preferred embodiment, the process temperature during oxidative processing is maximum 150° C., preferably maximum 140° C., more preferably maximum 130° C., and with the highest preference maximum 120° C. During oxidative processing, the agglomerated bodies are boiled. Said boiling is preferably carried out by first dissolving the agglomerated bodies in water and subsequent boiling of said mixture of water and agglomerated bodies.

In a further embodiment, the agglomerated bodies are dissolved in water in a ratio by weight between 1/5 and 1/15, preferably between 1/6 and 1/14, more preferably between 1/7 and 1/13, even more preferably between 1/8 and 1/12, even more preferably between 1/9 and 1/11, most preferably about 1/10.

In a preferred embodiment, oxidative processing is carried out for a duration of maximum 5 hours, preferably maximum 4.5 hours, more preferably maximum 4 hours, even more preferably maximum 3.5 hours, and most preferably maximum 3 hours.

In a another or further preferred embodiment, oxidative processing is carried out for a duration between 10 min and 5 hours, preferably between 20 min and 4 hours, and most preferably between 0.5 hours and 3 hours.

In a preferred embodiment, said oxidative processing is carried out under pressure, preferably said pressure is an overpressure of maximum 15 bar, preferably maximum 10 bar.

In a preferred embodiment, said oxidative processing is carried out in an enclosed chamber provided with a head space comprising a gas mixture. In a further embodiment at least a part of said gas mixture in said head space is replaced by air at least once.

It has been found that replacing at least part of said head space gas mixture with air improves oxidative processing. Replacing the head space gas mixture solves the issue of poor mass transfer between liquid (lignin) and gas phase (oxygen). The term “air” refers to ambient air comprising between 18 and 22 percent oxygen gas by volume, in m³ oxygen gas per m³ ambient air.

Alternatively, at least a part of said gas mixture in said head space is replaced by pure oxygen at least once. Pure oxygen gas is envisioned to be oxygen gas that is at least 95% pure, preferably at least 96% pure, more preferably at least 97% pure, even more preferably at least 98% pure, even more preferably at least 99% pure.

In a preferred embodiment, at least 50% by volume of the gas mixture in the head space is replaced by air or pure oxygen at least once, preferably at least 60% by volume, more preferably at least 70% by volume, even more preferably at least 80% by volume, even more preferably at least 90% by volume.

Preferably, said gas mixture is replaced multiple times during oxidative processing, for example 2, 3, 4, or 5 times.

In some embodiments, prior to oxidative processing a decantation/filtration step can be introduced wherein the agglomerated bodies are first steeped in water to extract soluble lignin. The obtained liquor is then transferred to the oxidative processing (step ii).

There are three monolignol monomers that are methoxylated to various degrees: p-coumaryl alcohol, coniferyl alcohol, and sinapyl alcohol. These lignols are incorporated into lignin in the form of the phenylpropanoids p-hydroxyphenyl (H), guaiacyl (G), and syringyl (S), respectively. The type and amount of phenolic aldehydes obtainable from the lignocellulosic biomass are dependent on the type and/or amount of a particular type of lignin in the biomass (i.e., H, G, and/or S). That is, the available percentage of precursors in the lignin structure strictly determines the formation of phenolic aldehydes.

In any of the embodiments disclosed and/or claimed herein, the phenolic aldehydes may comprise at least one of vanillin, syringaldehyde, apocynin, vanillic acid, acetosyringenone and syringic acid. In a preferred embodiment the phenolic aldehydes comprise vanillin, apocynin, vanillic acid, or a mixture thereof.

In any of the embodiments disclosed and/or claimed herein, the compounds obtainable from, preferably obtained from, the lignocellulosic biomass comprise at least one compound selected from the list of: vanillin, syringaldehyde, apocynin, vanillic acid, acetosyringone, syringic acid and phenolic aldehydes. In a preferred embodiment, the compounds obtainable from, preferably obtained from, the lignocellulosic biomass comprise at least one compound selected from the list of: vanillin, syringaldehyde, apocynin, vanillic acid, acetosyringone and syringic acid. In a more preferred embodiment the compounds obtainable from, preferably obtained from, the lignocellulosic biomass comprise at least one compound selected from the list of: vanillin, apocynin, vanillic acid, or a mixture thereof.

In a preferred embodiment, the agglomerated bodies formed during agglomeration comprises between 0.01 and 10% by weight of total amount of phenolic aldehydes, preferably between 0.025 and 7.5% by weight of total amount of phenolic aldehydes, more preferably between 0.05 and 5% by weight of total amount of phenolic aldehydes.

In a preferred embodiment, the pH is maintained above or equal to 12 during oxidative processing. This can be achieved by adding bases to the boiling mixture.

In a preferred embodiment, said boiling results in a solid and a liquid phase. In a further embodiment, said solid and liquid phase are separated. The phenolic aldehydes are obtained in the liquid phase. In a preferred embodiment, the method comprises further the step of isolation of the phenolic aldehydes from the liquid phase obtained from the biomass treatment. Isolation can be achieved by techniques known in the art, such as liquid-liquid extraction with an organic solvent, crystallization, and/or evaporation.

In a first preferred embodiment, the phenolic aldehydes are extracted in an organic phase and subjected to evaporation. After evaporation, a crude vanillin powder is purified by re-crystallization.

In another embodiment, a crude phenolic aldehyde powder or oil is obtained by re-crystallization. More preferably, a crude vanillin powder or oil is obtained by re-crystallization.

In a second preferred embodiment, the phenolic aldehydes are extracted in an organic phase, followed by re-uptake in water and crystallization via controlled acidifying.

In a particularly preferred embodiment, the method comprises the steps of:
i. mechanochemically reacting said lignocellulosic biomass with a dry inorganic oxide, a dry inorganic hydroxide, or a mixture thereof, thereby agglomerating said lignocellulosic biomass with said dry inorganic oxide, said dry inorganic hydroxide, or said mixture thereof, under pressure to form agglomerated bodies; and
ii. oxidative processing of the agglomerated bodies, comprising the step of boiling said agglomerated bodies for a period of time sufficient to extract phenolic aldehydes from said agglomerated bodies, wherein said lignocellulosic biomass is derivable from one or more herbaceous crops, and wherein the phenolic aldehydes comprises at least one of vanillin, apocynin, vanillic acid, syringaldehyde, acetosyringone, syringic acid, or a mixture thereof.

In another particularly preferred embodiment, the method comprises the steps of:
i. mechanochemically reacting said lignocellulosic biomass a dry inorganic oxide, a dry inorganic hydroxide, or a mixture thereof, thereby agglomerating said lignocellulosic biomass with said dry inorganic oxide, said dry inorganic hydroxide, or said mixture thereof, under pressure to form agglomerated bodies; and
ii. oxidative processing of the agglomerated bodies, comprising the step of boiling said agglomerated bodies for a period of time sufficient to extract phenolic aldehydes from said agglomerated bodies, wherein said lignocellulosic biomass is flax, straw, or a combination thereof.

In a most preferred embodiment, the method comprises the steps of:
i. mechanochemically reacting said lignocellulosic biomass a dry inorganic oxide, a dry inorganic hydroxide, or a mixture thereof, thereby agglomerating said lignocellulosic biomass with said dry inorganic oxide, said dry inorganic hydroxide, or said mixture thereof, under pressure to form agglomerated bodies; and
ii. oxidative processing of the agglomerated bodies, comprising the step of boiling said agglomerated bodies for a period of time sufficient to extract phenolic aldehydes from said agglomerated bodies, wherein said lignocellulosic biomass is flax, straw, or a combination thereof, and wherein the phenolic aldehydes comprises at least one of vanillin, apocynin, vanillic acid, syringaldehyde, acetosyringone, syringic acid, or a mixture thereof. In a second aspect the invention relates to phenolic aldehydes which are extracted non-catalytically.

The phenolic aldehydes are preferably extracted according to the method according to the first aspect of the invention.

In a third aspect, the invention provides a method for pretreating a lignocellulosic biomass. More specifically, the invention provides a method for pretreating a lignocellulosic biomass suitable for subsequent non-catalytic extraction of phenolic aldehydes from said lignocellulosic biomass.

In a particularly preferred embodiment, the invention provides a method for pretreating a lignocellulosic biomass, comprising the step of agglomerating said lignocellulosic biomass with a dry base under pressure to form one or more agglomerated bodies, wherein said dry base is a dry inorganic oxide, a dry inorganic hydroxide, or a mixture thereof.

The inventors have unexpectedly found that when a dry inorganic oxide, a dry inorganic hydroxide, or a mixture thereof, is combined with a lignocellulosic material and agglomerated in a non-aqueous and solvent-free environment, a high yield of phenolic aldehydes, such as vanillin, syringaldehyde, acetosyringone, vanillic acid, apocynin, and syringic acid can be released from the lignocellulosic biomass.

In an embodiment, said dry base is a dry alkali. In a preferred embodiment, said dry base is chosen from the list of: dry sodium hydroxide (NaOH), dry potassium hydroxide (KOH), dry calcium hydroxide (Ca(OH)₂), dry calcium oxide (CaO), dry magnesium hydroxide (Mg(OH)₂), dry magnesium oxide (MgO), or a mixture thereof, preferably dry NaOH, dry KOH, or a mixture thereof.

Alternatively, an inorganic carbonate can be used for agglomeration with the lignocellulosic biomass, such as sodium carbonate (Na₂CO₃) or potassium carbonate (K₂CO₃).

A significant advantage of the presently disclosed and/or claimed inventive concept(s) is that the processes described herein can be performed at ambient temperature without the need for added heat, cooling, or modifying pressure. Instead, the agglomeration can be performed under ambient conditions.

Without wishing to be bound by theory, it is believed the agglomeration step allows more of the lignocellulosic biomass to come into contact with the base. Even further, it is believed that the heat created by the agglomeration step facilitates the depolymerization of the lignin by increasing the rate of oxidative cleavage.

The method as described herein can also be used to extract phenolic aldehydes directly from the lignocellulosic biomass, without the need for subsequent extraction. To this end, after the step of agglomeration, the phenolic aldehydes may be separated via a separating step in order to provide individual compounds (i.e., the phenolic aldehydes) which may be quantitated and/or used in the preparation of other chemicals of interest. Any suitable method of determining the amount of phenolic aldehyde may be used, such as by chromatographic methods well known in the art. Moreover, the presence of particular phenolic aldehydes may be confirmed by any suitable chromatography method, such as thin-layer chromatograph, gas chromatography (GC), high-pressure liquid chromatography (HPLC), GC-MS, LC-MS, or any other suitable method known in the art. The phenolic aldehydes may be separated out individually and stored. Alternatively, at least a portion of the phenolic aldehydes may be sent to a subsequent processing step prior to separating out individual phenolic aldehydes from one another. In either event, one or more of the phenolic aldehydes (either individually or in a mixture) may be sent to a secondary process to convert the phenolic aldehydes into secondary products. For example, but not by way of limitation, such secondary products may comprise seal swelling agents, biofuel additives, food and nutraceutical additives, flavoring agents, specialty chemical precursors, antibacterial agents, and other types of pharmaceutical and medicinal compounds.

The method according to the present invention is advantageous because the production time needed for agglomeration is low and biomass agglomeration equipment is easily scalable. In a preferred embodiment, the step of agglomeration to form an agglomerated body leads to a processing capacity of at least 100 kg per hour and may be scaled up to 12 metric tonnes per hour.

In a particularly preferred embodiment, the method comprises the step of agglomerating said lignocellulosic biomass with a dry inorganic oxide, a dry inorganic hydroxide, or a mixture thereof, under pressure to form one or more agglomerated bodies, wherein said lignocellulosic biomass is flax, straw, or a combination thereof.

In another particularly preferred embodiment, the method is suitable for subsequent extraction of phenolic aldehydes from said lignocellulosic biomass comprises the step of agglomerating said lignocellulosic biomass with a dry inorganic oxide, a dry inorganic hydroxide, or a mixture thereof, under pressure to form one or more agglomerated bodies, wherein said lignocellulosic biomass is derivable from one or more herbaceous crops, and wherein the phenolic aldehydes comprises at least one of vanillin, apocynin, vanillic acid, syringaldehyde, acetosyringone, syringic acid, or a mixture thereof.

In a most preferred embodiment, the method is suitable for subsequent extraction of phenolic aldehydes from said lignocellulosic biomass and comprises the step of agglomerating said lignocellulosic biomass with a dry inorganic oxide, a dry inorganic hydroxide, or a mixture thereof, under pressure to form one or more agglomerated bodies, wherein said lignocellulosic biomass is flax, straw, or a combination thereof, and wherein the phenolic aldehydes comprises at least one of vanillin, apocynin, vanillic acid, syringaldehyde, acetosyringone, or a mixture thereof.

In a fourth aspect, the invention relates to a use of a method according to the third aspect for extracting phenolic aldehydes from lignocellulosic biomass.

In one embodiment, the method can be used as a pretreatment step in an extraction process, wherein the agglomerated bodies serve as semi-fabricates.

In another embodiment, the method can be used to extract phenolic aldehydes directly from the lignocellulosic biomass, without the need for subsequent extraction.

In a fifth aspect, the invention relates to an agglomerated body obtained from a method according to the third aspect.

In a particularly preferred embodiment, said body comprises between 0.01 and 10% by weight of total amount of phenolic aldehydes, preferably between 0.025 and 7.5% by weight of total amount of phenolic aldehydes, more preferably between 0.05 and 5% by weight of total amount of phenolic aldehydes.

The invention is further described by the following non-limiting examples which further illustrate the invention, and are not intended to, nor should they be interpreted to, limit the scope of the invention.

### EXAMPLES

### Comparative Example 1

Flax shives (50 g) with a moisture content of 15% by weight are transferred to the inlet of a flat die pelletizing machine, equipped with 2 mechanical rollers. The metal die plate contains holes of 6 mm diameter and die thickness is 3 cm. A cutting blade installed under the die plate cuts pellets of at a length of between 1 and 2 cm. The pellets obtained are green and have a temperature of about 50° C. when leaving the pelletizer. Upon storage, the pellets stay intact, without color change.

### Example 2

Flax shives (50 g) with a moisture content of 15% by weight are homogenized with 40 g of sodium hydroxide pellets, to obtain an even distribution of sodium hydroxide pellets. This mixture is transferred to the inlet of a flat die pelletizing machine and pellets are produced in the manner as described for comparative example 1. The pellets obtained are rust brown and have a distinct smell reminiscent of amine compounds. The surface temperature of the pellets is between 85° C. and 100° C. when leaving the pelletizer.

### Example 3

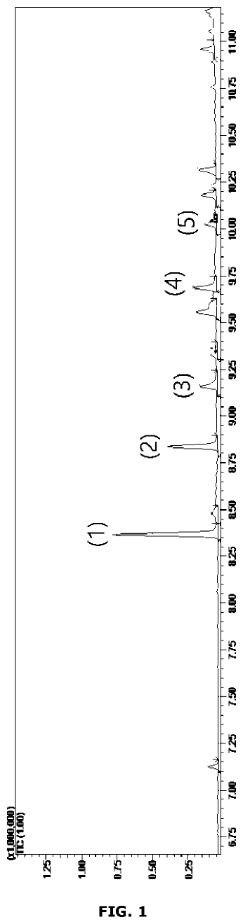

The obtained pellets from example 2 (1 g) were dispersed in water and the alkali content was neutralized with a 2 M hydrochloric acid solution. The organic compounds were extracted from 1 mL of the neutralized solution using an organic solvent such as ethyl acetate (1.5 mL). GC-MS analysis shows that phenolic compounds were released from the flax lignin fraction, mostly vanillin and derivatives thereof, such as apocynin, and vanillic acid, and syringaldehyde and derivatives thereof such as acetosyringone, and syringic acid. To better exemplify reference is made to FIG. 1, which shows a GC-MS chromatogram of an alkaline flax pellet obtained through the method of example 2, wherein the following references refer to:

| | | Relative |
|---|---|---|
| | Compound | composition (%) |
| (1) | Vanillin | 29.4 |
| (2) | Apocynin | 19.2 |
| (3) | Vanillic acid | 16.0 |
| (4) | Syringaldehyde | 7.3 |
| (5) | Acetosyringone | 8.3 |

Spiking of the organic phase with known amounts of vanillin indicates that 0.05 to 0.1% of vanillin is formed with respect to the biomass (flax shives) weight.

### Example 4

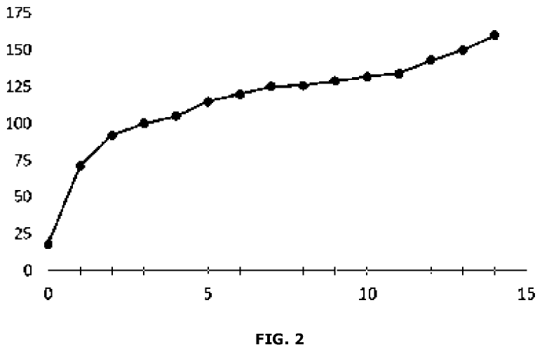

Flax shives are treated with sodium hydroxide as described in example 2. The pellets that are collected are re-introduced in the pelleting machine. With each cycle of pelleting, the pellets obtained become darker and temperature of the pellets raises up to final temperature of >150° C. when leaving the pelletizer. At that point, charring starts, and the pellet die clogs. At temperature above 150° C., the charring gives significant delignification and decomposition starts to occur. To better exemplify reference is made to FIG. 2, which shows a surface temperature profile (in ° C.) of pellets during repetitive pelleting (number of cycles).

### Example 5

The obtained pellets from example 4 (1 g) were prepared and analyzed as described in example 3. GC-MS analysis shows that phenolic compounds were released from the flax lignin fraction, but with more formation of various low-molecular weight products, aromatic and non-aromatic than in example 3. Major compounds found are apocynin (acetovanillone), homovanillyl alcohol, and vanillic acid. Non-phenolic compounds are lactic acid and 2-methoxypropanol. Vanillin is also detected.

### Example 6

Flax shives (50 g) with a moisture content of 15% by weight are homogenized with 20 g of sodium hydroxide pellets, to obtain an even distribution of sodium hydroxide pellets. This mixture is transferred to the inlet of a flat die pelletizing machine and pellets are produced in the manner as described for the example 1. The pellets obtained are rust brown and have a distinct smell reminiscent of amine compounds. The surface temperature of the pellets is between 85° C. and 90° C. when leaving the pelletizer.

Upon storage, the pellets gradually grow paler and start to disintegrate. In final stage, full disintegration to brown powder has occurred.

### Example 7

Flax shives (50 g) with a moisture content of 15% by weight are homogenized with 10 g of sodium hydroxide pellets, to obtain an even distribution of sodium hydroxide pellets. This mixture is transferred to the inlet of a flat die pelletizing machine and pellets are produced in the manner as described for the example 1. The pellets obtained are rust brown and have a distinct smell reminiscent of amine compounds. The surface temperature of the pellets is around 85° C. when leaving the pelletizer.

Upon storage, the pellets gradually become brown with green hue and start to disintegrate. In final stage, full disintegration to brown powder has occurred.

### Example 8

Flax shives (50 g) with a moisture content of 15% by weight are homogenized with 40 g of potassium carbonate powder (K₂CO₃), to obtain an even distribution of potassium carbonate. This mixture is transferred to the inlet of a flat die pelletizing machine and pellets are produced in the manner as described for comparative example 1. The pellets obtained are dark brown, but no smell of reminiscent of amine compounds is detected. The surface temperature of the pellets is between 85° C. and 100° C. when leaving the pelletizer.

### Example 9

The obtained pellets from example 8 (1 g) were prepared and analyzed as described in example 3. GC-MS analysis shows that practically no phenolic compounds were released from the flax lignin fraction.

### Example 10

Flax shives (50 g) with a moisture content of 15% by weight are homogenized with 40 g of calcium hydroxide, to obtain an even distribution of calcium hydroxide. This mixture is transferred to the inlet of a flat die pelletizing machine and pellets are produced in the manner as described for comparative example 1. The pellets obtained are dark green. The surface temperature of the pellets is between 95° C. and 120° C. when leaving the pelletizer.

Upon storage, the pellets remained intact and did not pulverize.

### Example 11

Flax shives (50 g) with a moisture content of 15% by weight are homogenized with 40 g of calcium oxide, to obtain an even distribution of calcium hydroxide. This mixture is transferred to the inlet of a flat die pelletizing machine and pellets are produced in the manner as described for comparative example 1. The pellets obtained are light green. The surface temperature of the pellets is >100° C. when leaving the pelletizer.

After 2 days of storage, the pellets completely disintegrated to form a pale, gray powder. Upon addition to water, a light brown slurry was obtained that was neutralized to pH 7 using sulfuric acid (96%). After evaporation of water, a paste was obtained.

### Example 12

Vanillin formation was investigated in the pellets obtained from examples 2, 6, and 7, and in the pellet obtained in comparative example 1. Therefore, the pellets were prepared and analyzed as described in example 3. Changes in vanillin formation was assessed by overlay of the different GC-MS chromatograms.

In the pellet obtained in comparative example 1, practically no vanillin was detected, while alkaline pellets (examples 2, 6, and 7) showed a clear presence of vanillin. Vanillin formation doubled when increasing alkali loading from flax/NaOH 5:1 (example 7) to flax/NaOH 5:2 (example 6). Vanillin formation doubled again when increasing alkali loading from flax/NaOH 5:2 to flax/NaOH 5:4 (example 2). The highest alkaline level (flax/NaOH 5:4) gave a series of side products as described in example 3.

### Example 13

Vanillin formation was investigated in the pellets obtained from examples 2 and 8. Therefore, the pellets were prepared and analyzed as described in example 3. Changes in vanillin formation was assessed by overlay of the different GC-MS chromatograms.

In the pellet from example 8, with potassium carbonate (flax/K₂CO₃ 5:4), vanillin formation was much weaker than in the pellet from example 2, with sodium hydroxide (flax/NaOH 5:4).

### Example 14

Pellets obtained from examples 2, 6, and 7, and comparative example 1 were added to demineralized water. Dispersion of the pellets with alkaline additive was immediate and a dark brown liquor was obtained within 5 minutes. Dispersion of the pellets without additive was slow and ineffective: pellets remain intact and compact and very few liquor formation is observed

### Example 15

About 300 mg of pellet from example 2 is dissolved in 1 M of an aqueous NaOH solution (3.5 mL) in a pressure tube. After sealing the tube, temperature is increased to 120° C. during stirring. After 48 h, reaction temperature is lowered to room temperature and the tube is opened. The mixture is centrifuged, and 1 mL of supernatant is taken and 1 mL of a 2 M HCl solution is added. Then, 1 mL of ethyl acetate (containing internal standard is added). After shaking, the organic phase is collected and analyzed by GC-MS.

Liquid-liquid extraction gave a relatively pure vanillin fraction. Without further purification efforts, flavor perception of this fraction was described as warm, sweet, and rich with notes of vanilla and caramel.

It is supposed that the present invention is not restricted to any form of realization described previously and that some modifications can be added to the presented example of fabrication without reappraisal of the appended claims. For example, the present invention has been described referring to flax shives, but it is clear that the invention can be applied to any herbaceous crop for instance or to with other forms of agglomerating, such as (co-)extrusion.

1. A method for extracting phenolic aldehydes from a lignocellulosic biomass comprising:
mechanochemically reacting said lignocellulosic biomass with a dry base, thereby agglomerating said lignocellulosic biomass with said dry base under pressure to form agglomerated bodies; and
oxidative processing of the agglomerated bodies, comprising boiling said agglomerated bodies for a period of time sufficient to extract phenolic aldehydes from said agglomerated bodies,
wherein said lignocellulosic biomass is derivable from one or more herbaceous crops, and wherein said dry base is a dry inorganic oxide, a dry inorganic hydroxide, or a mixture thereof.

2. The method according to claim 1, wherein said oxidative processing is carried out at a temperature of maximum 120° C.

3. The method according to claim 1, wherein said lignocellulosic biomass is reacted mechanochemically with said dry inorganic oxide, said dry inorganic hydroxide, or said mixture thereof, in a ratio by weight between 5/1 and 5/3.

4. The method according to claim 1, wherein said oxidative processing is carried out for a duration between 0.5 hours and 3 hours.

5. The method according to claim 1, wherein said oxidative processing is carried out in an enclosed chamber provided with a head space comprising a gas mixture, and wherein at least a part of said gas mixture in said head space is replaced by air at least once.

6. The method according to claim 1, wherein the surface temperature of said agglomerated bodies is higher than 90° C.

7. The method according to claim 1, wherein said phenolic aldehydes are vanillin, apocynin, vanillic acid, syringic acid, syringaldehyde, acetosyringone, or a mixture thereof.

8. The method according to claim 1, wherein said lignocellulosic biomass is flax, straw, or a mixture thereof.

9. The method according to claim 1, wherein during said agglomerating said lignocellulosic biomass and said dry base are pelletized into rounded, spherical, or cylindrical pellets.

10. The method according to claim 1, wherein during said agglomerating said lignocellulosic biomass and said dry base are co-extruded.

11. The method according to claim 1, wherein said inorganic oxide, inorganic hydroxides, or said mixture thereof are chosen from dry sodium hydroxide (NaOH), dry potassium hydroxide (KOH), dry calcium hydroxide (Ca(OH)₂), dry calcium oxide (CaO), dry magnesium hydroxide (Mg(OH)₂), dry magnesium oxide (MgO), or a mixture thereof.

12. The method according to claim 1, wherein the lignocellulosic biomass has a moisture content between 8 and 15% by weight prior to performing the method.

13. The method according to claim 1, wherein the mechanochemically reacting is repeated at least once.

14. Phenolic aldehydes, characterized in that the phenolic aldehydes are extracted non-catalytically.

15. The method according to claim 11, wherein said inorganic oxide, inorganic hydroxides, or said mixture thereof is dry NaOH, dry KOH, or a mixture thereof.

16. The method according to claim 13, wherein the mechanochemically reacting is repeated twice.
